(19) United States
(12) Patent Application Publication
(10) Pub. No.: US 2017/0189268 A1
(43) Pub. Date: Jul. 6, 2017

(54) APPARATUS, SYSTEM AND METHOD FOR REMOTELY DETERMINING ACUPUNCTURE POINT

(71) Applicant: BOE TECHNOLOGY GROUP CO., LTD., Beijing (CN)

(72) Inventors: Dong CHAI, Beijing (CN); Hong WANG, Beijing (CN); Jian SONG, Beijing (CN); Linzhe LI, Beijing (CN); Xuewen LV, Beijing (CN)

(21) Appl. No.: 15/031,457

(22) PCT Filed: Nov. 11, 2015
(86) PCT No.: PCT/CN2015/094247

§ 371 Date: Apr. 22, 2016

(30) Foreign Application Priority Data
May 22, 2015 (CN) 201510268800.6

(51) Int. Cl. A61H 39/02; A61B 90/13; A61B 5/00

(52) U.S. Cl. CPC A61H 39/02; A61B 5/0077; A61B 90/13; A61B 5/4854; A61B 2576/00

(57) ABSTRACT

The disclosure provides an apparatus, system and method for remotely determining acupuncture point. The apparatus comprises a first controller, an image acquisition module, a first image transport module and a positioning mark generation module, the image acquisition module used for acquiring a target image of a part of the body of a target person on which the acupuncture point is required to be determined; the first image transport module used for transmitting the target image to a remote device; the first image transport module further used for transmitting the received target image marked with a first mark to the first controller; the first controller used for determining a position coordinate of the acupuncture point according to the target image marked with the first mark and controlling the positioning mark generation module to generate a second mark at the position coordinate of the acupuncture point according to the position coordinate.

### CROSS REFERENCE TO RELATED APPLICATIONS

This application claims priority to China patent application number 201510268800.6, which was filed on May 22, 2015, the disclosure of which is incorporated by reference herein in its entirety.

### BACKGROUND

The disclosure relates to apparatus and system for remotely determining acupuncture point.

The determination of acupuncture point has a significant impact on the treatment of diseases when curing some diseases. A professional doctor is required to determine the acupuncture point on a target person's body, and then applies acupuncture at the acupuncture point to cure some diseases. A person without professional training cannot determine the position of the acupuncture point to be acupunctured on the target person's body, therefore the target person needs to go to hospital when the acupuncture point of the target person is required to be determined, which may be very inconvenient for the target person unable to move freely.

Therefore, how to facilitate the target person unable to move freely to determine the acupuncture point on his body has become a technical problem to be solved in the field.

### BRIEF DESCRIPTION

The disclosure aims at providing an apparatus for remotely determining acupuncture point and a system including the apparatus. Ordinary nursing personnel can accurately determine the position of the acupuncture point by utilizing the system, therefore the target person does not have to go to hospital, and the acupuncture point on the body of the target person can be determined by the ordinary nursing personnel at home.

There is provided an apparatus for remotely determining acupuncture point according to an aspect of the disclosure. The apparatus comprises a first controller, an image acquisition module, a first image transport module and a positioning mark generation module, wherein the image acquisition module is used for acquiring a target image of a part of the body of a target person on which the acupuncture point is required to be determined; the first image transport module is used for transmitting the target image to a remote device; the first image transport module is further used for transmitting the received target image marked with a first mark to the first controller; the first controller determining a position coordinate of the acupuncture point according to the target image marked with the first mark and controlling the positioning mark generation module to generate a second mark at the position coordinate of the acupuncture point according to the position coordinate.

According to an embodiment, the position coordinate of the acupuncture point is a three-dimensional coordinate, and the first controller comprises a control unit and a three-dimensional coordinate conversion unit, wherein the control unit can control the three-dimensional coordinate conversion unit to convert a two-dimensional coordinate of the first mark in the target image marked with the first mark to the three-dimensional coordinate within a space in which the target person is located.

According to an embodiment, the image acquisition module can further acquire in real time a reference image of the part of the body of the target person on which the acupuncture point is required to be determined, the first controller further comprises a deciding unit, wherein the deciding unit is used for deciding whether the reference image is consistent with the target image and sending a signal indicating consistency to the controller unit when the reference image and the target image are consistent, the controller unit, after receiving the signal indicating consistency, sends a signal indicating start of conversion to the three-dimensional coordinate conversion unit, and the three-dimensional coordinate conversion unit converts the two-dimensional coordinate of the first mark to the three-dimensional coordinate after receiving the signal indicating start of conversion.

According to an embodiment, the first controller further comprises a relationship transformation unit, wherein the deciding unit sends a signal indicating inconsistency to the controller unit when the reference image is inconsistent with the target image, the controller unit, after receiving the signal indicating inconsistency, can send a signal indicating start of transformation to the relationship transformation unit which determines a relationship between a two-dimensional coordinate system in the target image and a three-dimensional space in which the target person is located according to the signal indicating start of transformation, and the three-dimensional coordinate conversion unit obtains the three-dimensional coordinate of the second mark according to the relationship.

According to an embodiment, the positioning mark generation module comprises a drive motor and a light pointer, wherein the light pointer can emit an optical signal, a controlling end of the drive motor is connected with the first controller, the drive motor, after receiving the position coordinate of the acupuncture point, can drive the light pointer to move to cause the light pointer to generate the second mark at the position of the acupuncture point.

According to an embodiment, the light pointer comprises projector or laser pointer.

There is provided a system for remotely determining acupuncture point according to another aspect of the disclosure. The system comprises the above mentioned apparatus for remotely determining acupuncture point provided by the disclosure and a remote device, wherein the remote device comprises a second image transport module for communicating with the first image transport module and a mark writing module for adding the first mark on the received target image.

According to an embodiment, the first image transport module can send the target image acquired by the image acquisition module to the second image transport module which is used for receiving the target image and can send the target image with the first mark to the first image transport module.

The nursing personnel unable to determine the position of the acupuncture point can also accurately determine the acupuncture point of the target person by utilizing the apparatus and system provided by the disclosure, therefore the target person does not have to leave their lodgings and go to hospital.

### BRIEF DESCRIPTION OF THE DRAWINGS

The accompanying drawings is provided for further understanding the present invention, constitutes a part of the specification and is used for explaining the present invention in connection with embodiments, but is not aimed to limit the disclosure, in which:

FIG. 1 schematically shows a block diagram of an apparatus for remotely determining acupuncture point in accordance with an embodiment of the disclosure; and

FIG. 2 shows a schematic diagram of a system for remotely determining acupuncture point in accordance with an embodiment of the disclosure.

### DETAILED DESCRIPTION

To make those skilled in the art to better understand the objects, technical solutions and advantages of embodiments of the disclosure, the technical solution of embodiments of the disclosure will be described clearly and completely in connection with the accompanying drawings of embodiments of the disclosure. It is obvious that the described embodiments are only some, but not all the embodiments of the disclosure. All the other embodiments made by those of ordinary skill in the art without the premise of creative work belong to the scope of the disclosure.

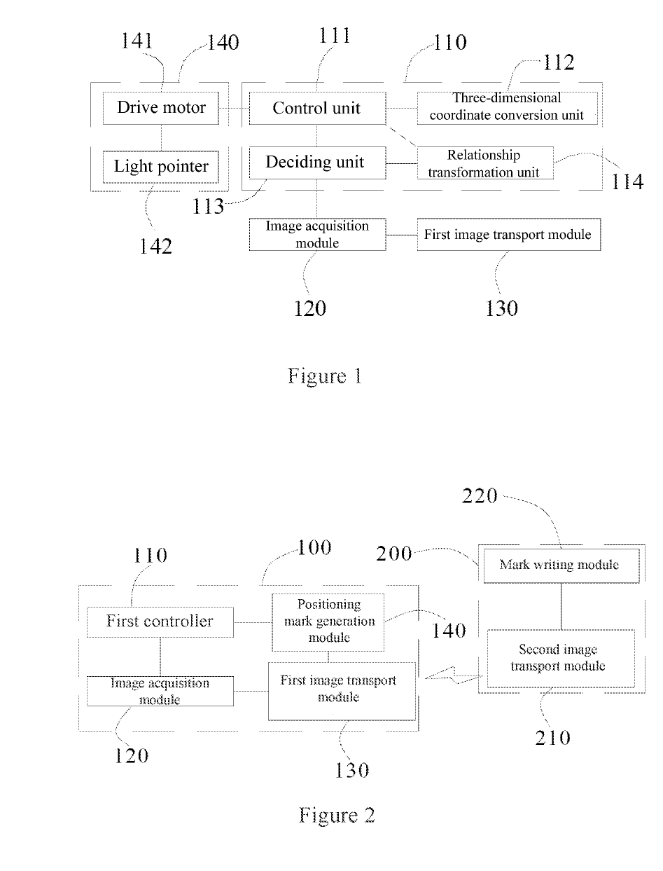

As shown in FIG. 1 and FIG. 2, as an embodiment of the disclosure, there is provided an apparatus for remotely determining acupuncture point. The apparatus includes a first controller 110, an image acquisition module 120, a first image transport module 130 and a positioning mark generation module 140.

The image acquisition module 120 is used for acquiring a target image of a part of the body of a target person on which the acupuncture point is required to be determined. The image acquisition module 120 may acquire a two-dimensional target image and/or a three-dimensional target image. The first image transport module 130 is used for transmitting the target image acquired by the image acquisition module 120 to a remote device 200.

In the disclosure, the apparatus for remotely determining acupuncture point is arranged at a side of the target person, and the apparatus 100 is operated by the nursing personnel at the side of the target person or by the target person. The disclosure does not make the specific stipulation to the concrete structure of the image acquisition module 120, for example, the image acquisition module 120 may be an image acquisition apparatus such as a high definition camera or the like. The apparatus 100 may be operated by the nursing personnel at the side of the target person, for example, the nursing personnel, after starting the apparatus 100, can utilize the image acquisition module 120 to take a picture of a part of the body of the target person to be acupunctured to obtain the target image. It can be easily understood that the nursing personnel, before utilizing the image acquisition module 120 to acquire the target image, needs to communicate with a doctor and informs the doctor of the specific condition of the target person, and the doctor decides which acupuncture points of the target person are required to be treated and then informs the nursing personnel of the approximate positions of the acupuncture points on a human body. The first image transport module 130 is used for transmitting the target image to the remote device 200 after the image acquisition module 120 acquires the target image. The doctor at a side of the remote device 200 analyses the target image received by the remote device 200 to determine the exact position of the acupuncture point to be acupunctured, and then marks the exact position (i.e., the first mark) of the acupuncture point on the target image. The exact position may be corresponding to a two-dimensional coordinate or a three-dimensional coordinate depending on whether the target image is a two-dimensional target image or a three-dimensional target image. The image marked with the position (i.e., the first mark) of the acupuncture point is transmitted to the apparatus 100 at the side of the target person.

The first image transport module 130 in the apparatus 100 is further used for transmitting the received target image marked with the first mark to the first controller 110 which determines a position coordinate of the acupuncture point according to the target image marked with the first mark and controls the positioning mark generation module 140 to generate a second mark at the position coordinate of the acupuncture point according to the position coordinate.

It is noted that the second mark herein is located on the body of the target person and the nursing personnel applies acupuncture at the second mark.

The nursing personnel unable to determine the position of the acupuncture point can also accurately determine the acupuncture point of the target person through the above description, therefore the target person does not have to leave their lodgings and go to hospital.

In an embodiment, the position coordinate of the acupuncture point determined by the first controller 110 is a three-dimensional coordinate, and the positioning mark generation module 140 may form a positioning mark at the three-dimensional coordinate representing the position of the acupuncture point. In an embodiment, the target image and the target image marked with the first mark are transmitted in a way of two-dimensional image, in other words, the doctor at the side of the remote device would mark the first mark on a two-dimensional target image to form a new two-dimensional image, and the remote device 200 would transmit the new two-dimensional image to the apparatus 100. In order to form the second mark within a three-dimensional space in which the target person is located, the first controller 110 may include a control unit 111 and a three-dimensional coordinate conversion unit 112 as shown in FIG. 1. The control unit 111 can control the three-dimensional coordinate conversion unit 112 to convert a two-dimensional coordinate of the first mark in the target image marked with the first mark to the three-dimensional coordinate within a space in which the target person is located. The control unit 111 further controls the positioning mark generation module 140 to form the second mark at the above mentioned three-dimensional coordinate.

In general, a person cannot maintain a same action for a long time. In other words, the target person may not maintain the posture in the target image any more when the target image marked with the first mark is transmitted to the side of the target person.

In an embodiment, a real time posture of the target person can also be decided after the target image marked with the first mark is received. When the real time posture of the target person is consistent with the posture in the target image after the target image marked with the first mark is received, a two-dimensional coordinate of the first mark in the target image can be directly converted to a three-dimensional coordinate in a three-dimensional space in which the target person is located if the first mark is represented by the two-dimensional coordinate; a three-dimensional coordinate can be directly used if the first mark is represented by the three-dimensional coordinate. Correspondingly, the image acquisition module 120 can further acquire in real time a reference image of the part of the body of the target person on which the acupuncture point is required to be determined, wherein the reference image may be a two- or three-dimensional reference image. As shown in FIG. 1, the first controller 110 further includes a deciding unit 113, wherein the deciding unit 113 is used for deciding whether the reference image is consistent with the target image and sending a signal indicating consistency to the controller unit 111 when the reference image and the target image are consistent. The controller unit 111, after receiving the signal indicating consistency, may send a signal indicating start of conversion to the three-dimensional coordinate conversion unit 112 if the first mark is represented by a two-dimensional coordinate, which would convert the two-dimensional coordinate of the first mark to the three-dimensional coordinate after receiving the signal indicating start of conversion. If the first mark is represented by a three-dimensional coordinate, the three-dimensional coordinate can be directly used.

When the posture of the target person shown in the reference image is not consistent with the posture in the target image, the nursing personnel may adjust the posture of the target person such that the target person may return to the posture shown in the target image.

For the convenience of the target person, the target image marked with the first mark instead of the posture of the target person may be adjusted. In an embodiment, the first controller 110 may further includes a relationship transformation unit 114, wherein the deciding unit 113 sends a signal indicating inconsistency to the control unit 111 which can send a signal indicating start of transformation to the relationship transformation unit 114 after receiving the signal indicating inconsistency, the relationship transformation unit 114, after receiving the signal indicating start of transformation, determines a relationship between a two-dimensional coordinate system in the target image and a three-dimensional space in which the target person is located if the target image is a two-dimensional target image, and the three-dimensional coordinate conversion unit obtains the three-dimensional coordinate of the second mark according to the relationship. If the target image is a three-dimensional target image, the three-dimensional coordinate conversion unit may determine the three-dimensional coordinate of the second mark according to the location movement between the target person in the target image and the target person in the reference image.

In an embodiment, the target image is a two-dimensional target image, and the relationship transformation unit 114 may analyze the muscle and body outline of the target person to obtain a transformation relationship between the target image marked with the first mark and the reference image when the target image marked with the first mark is adjusted, after this, the three-dimensional coordinate conversion unit converts the two-dimensional coordinate of the first mark to the three-dimensional coordinate of the second mark according to the above transformation relationship.

It can be easily understood that the three-dimensional coordinate conversion unit 112 uses the two-dimensional coordinate or the three-dimensional coordinate of the first mark in the adjusted target image to generate the three-dimensional coordinate of the second mark. It can be further convenient to the target person without the adjustment of posture of the target person by the arrangement of the relationship transformation unit 114.

There is not a particular requirement for the specific structure of the positioning mark generation module in the disclosure only if it can form the second mark at the above three-dimensional coordinate. The second mark may be an optical signal mark or other entity mark. When the second mark is an entity mark such as a small ball, the positioning mark generation module may further include a drive apparatus for driving the entity mark to the three-dimensional coordinate.

In an embodiment, for accurate positioning, the positioning mark generation module 140 may include a drive motor 141 and a light pointer 142, wherein the light pointer 142 can emit an optical signal, a controlling end of the drive motor 141 is connected with the first controller 110, the drive motor 141 can drive the light pointer 142 to move after receiving the position coordinate of the acupuncture point, to cause the light pointer 142 to generate the second mark at the position of the acupuncture point.

In this embodiment, the second mark may be a light spot projecting on the body of the target person. The smaller an area of the light spot is, the more accurate the position of the mark is. In an embodiment, in order to obtain a smaller light spot, the light pointer 141 may include projector or laser pointer.

Another aspect of the disclosure further provides a system for remotely determining acupuncture point. As shown in FIG. 2, the system includes the above mentioned apparatus 100 for remotely determining acupuncture point provided by the disclosure and a remote device 200, wherein the remote device 200 includes a second image transport module 210 for communicating with the first image transport module 120 and a mark writing module 220 for adding the first mark on the received target image.

The remote device 200 is operated by a professional doctor as described above, and may be a personal computer comprising a second controller for controlling the running and operation of the image transport module 210 and the mark writing module 220. The mark writing module 220 may be an input device such as keyboard, mouse, digital panel, or the like.

As mentioned above, in the system provided by the disclosure, the apparatus 100 may convert the position coordinate of the acupuncture point to the second mark in a three-dimensional space in which the target person is located after the doctor at the side of the remote device marks the position of the acupuncture point on the target image, wherein the second mark is on the body of the target person and the nursing personnel can apply acupuncture at the second mark. Therefore the system provided by the disclosure is especially suitable for determining the acupuncture point of the body of the target person unable to move freely.

In the disclosure, the apparatus 100 communicates with the remote device 200 through Internet.

A flowchart of determining the acupuncture point of the target person by utilizing the system provided by the disclosure is described in the following. In this embodiment, the image acquisition module is a high definition camera or any other suitable image acquisition device.

Utilizing the system provided by the disclosure to determine the acupuncture point may include the following steps:

acquiring a target image by utilizing the high definition camera;

transmitting the target image to a remote device, wherein the target image is viewed by a professional doctor at the side of the remote device;

when the professional doctor is able to determine the position of the acupuncture point through the target image, the professional doctor selects the position of the acupuncture point on the target image displayed by the remote device through a mouse pointer to form a first mark; when the professional doctor is unable to determine the position of the acupuncture point through the target image, the apparatus at the side of the patient is required to reacquire an image and send it to the remote device until the professional doctor can determine the position of the acupuncture point;

the doctor returns the target image marked with the first mark to the apparatus for remotely determining acupuncture point;

the target image marked with the first mark is compared with the a reference image to decide whether the postures of patient in the two images are same;

when the postures of patient in the two images are same, the two-dimensional coordinate of the first mark in the target image can be converted to the three-dimensional coordinate within a three-dimensional space in which the target person is located through the three-dimensional coordinate conversion unit; when the postures of patient in the two images are not same, the muscle and body outline of the target person is analyzed to obtain a relationship between the target image and the reference image through the relationship transformation unit, then the three-dimensional coordinate conversion unit determines the three-dimensional coordinate of the second mark according to the relationship;

the control unit controls the operation of the drive motor, thereby driving the light pointer to emit a light to form the second mark on the surface of the body of the target person, wherein the coordinate of the second mark is the three-dimensional coordinate as mentioned above;

finally, the nursing personnel applies acupuncture at the second mark.

It may be understood that the above mentioned embodiments are only exemplary embodiments for the illustration of the principle of the disclosure, however the disclosure is not intend to it. Various modifications and variations can be made by those skilled in the art without departing from the spirit and the scope of the disclosure, which are deemed to be within the scope of the disclosure.

1. An apparatus for remotely determining acupuncture point, comprising a first controller, an image acquisition module, a first image transport module and a positioning mark generation module,
the image acquisition module used for acquiring a target image of a part of the body of a target person on which the acupuncture point is required to be determined;
the first image transport module used for transmitting the target image to a remote device;
the first image transport module further used for transmitting the received target image marked with a first mark to the first controller;
the first controller determining a position coordinate of the acupuncture point according to the target image marked with the first mark, and controlling the positioning mark generation module to generate a second mark at the position coordinate of the acupuncture point according to the position coordinate.

2. The apparatus according to claim 1, wherein the position coordinate of the acupuncture point is a three-dimensional coordinate, and the first controller comprises a control unit and a three-dimensional coordinate conversion unit, wherein the control unit can control the three-dimensional coordinate conversion unit to convert a two-dimensional coordinate of the first mark in the target image marked with the first mark to the three-dimensional coordinate within a space in which the target person is located.

3. The apparatus according to claim 2, wherein the image acquisition module can further acquire in real time a reference image of the part of the body of the target person on which the acupuncture point is required to be determined, the first controller further comprises a deciding unit, wherein the deciding unit is used for deciding whether the reference image is consistent with the target image and sending a signal indicating consistency to the controller unit when the reference image and the target image are consistent, the controller unit, after receiving the signal indicating consistency, sends a signal indicating start of conversion to the three-dimensional coordinate conversion unit, and the three-dimensional coordinate conversion unit converts the two-dimensional coordinate of the first mark to the three-dimensional coordinate after receiving the signal indicating start of conversion.

4. The apparatus according to claim 3, wherein the first controller further comprises a relationship transformation unit, the deciding unit sends a signal indicating inconsistency to the controller unit when the reference image is inconsistent with the target image, the controller unit, after receiving the signal indicating inconsistency, can send a signal indicating start of transformation to the relationship transformation unit which determines a relationship between a two-dimensional coordinate system in the target image and a three-dimensional space in which the target person is located according to the signal indicating start of transformation, and the three-dimensional coordinate conversion unit obtains the three-dimensional coordinate of the second mark according to the relationship.

5. The apparatus according to claim 1, wherein the positioning mark generation module comprises a drive motor and a light pointer, the light pointer can emit an optical signal, a controlling end of the drive motor is connected with the first controller, the drive motor, after receiving the position coordinate of the acupuncture point, can drive the light pointer to move to cause the light pointer to generate the second mark at the position of the acupuncture point.

6. The apparatus according to claim 5, wherein the light pointer comprises projector or laser pointer.

7. A system for remotely determining acupuncture point, comprising the apparatus for remotely determining acupuncture point according to any one of claim 1 and a remote device, wherein the remote device comprises a second image transport module for communicating with the first image transport module and a mark writing module for adding the first mark on the received target image.

8. The system according to claim 7, wherein the first image transport module can send the target image acquired by the image acquisition module to the second image transport module which is used for receiving the target image and can send the target image with the first mark to the first image transport module.

9. The apparatus according to claim 2, wherein the positioning mark generation module comprises a drive motor and a light pointer, the light pointer can emit an optical signal, a controlling end of the drive motor is connected with the first controller, the drive motor, after receiving the position coordinate of the acupuncture point, can drive the light pointer to move to cause the light pointer to generate the second mark at the position of the acupuncture point.

10. The apparatus according to claim 3, wherein the positioning mark generation module comprises a drive motor and a light pointer, the light pointer can emit an optical signal, a controlling end of the drive motor is connected with the first controller, the drive motor, after receiving the position coordinate of the acupuncture point, can drive the light pointer to move to cause the light pointer to generate the second mark at the position of the acupuncture point.

11. The apparatus according to claim 4, wherein the positioning mark generation module comprises a drive motor and a light pointer, the light pointer can emit an optical signal, a controlling end of the drive motor is connected with the first controller, the drive motor, after receiving the position coordinate of the acupuncture point, can drive the light pointer to move to cause the light pointer to generate the second mark at the position of the acupuncture point.

12. A method for remotely determining acupuncture point, comprising:
acquiring a target image of a part of the body of a target person on which the acupuncture point is required to be determined;
transmitting the target image to a remote device;
receiving the target image marked with a first mark from the remote device;
determining a position coordinate of the acupuncture point according to the target image marked with the first mark; and
generating a second mark at the position coordinate of the acupuncture point according to the position coordinate.

13. The method according to claim 12, wherein the position coordinate of the acupuncture point is a three-dimensional coordinate, and the method further comprises:
converting a two-dimensional coordinate of the first mark in the target image marked with the first mark to the three-dimensional coordinate within a space in which the target person is located.

14. The method according to claim 13, further comprising:
acquiring in real time a reference image of the part of the body of the target person on which the acupuncture point is required to be determined;
deciding whether the reference image is consistent with the target image; and
converting the two-dimensional coordinate of the first mark to the three-dimensional coordinate when the reference image and the target image are consistent.

15. The method according to claim 14, further comprising:
determining a relationship between a two-dimensional coordinate system in the target image and a three-dimensional space in which the target person is located when the reference image is inconsistent with the target image; and
obtaining the three-dimensional coordinate of the second mark according to the relationship.

16. The method according to claim 12, further comprising:
driving a light pointer to move to cause the light pointer to generate the second mark at the position of the acupuncture point.

17. The method according to claim 13, further comprising:
driving a light pointer to move to cause the light pointer to generate the second mark at the position of the acupuncture point.

18. The method according to claim 14, further comprising:
driving a light pointer to move to cause the light pointer to generate the second mark at the position of the acupuncture point.

19. The method according to claim 15, further comprising:
driving a light pointer to move to cause the light pointer to generate the second mark at the position of the acupuncture point.

20. The method according to claim 16, wherein the light pointer comprises projector or laser pointer.
